(19) United States
(12) Patent Application Publication
(10) Pub. No.: US 2015/0359819 A1
(43) Pub. Date: Dec. 17, 2015

(54) TOPICAL FORMULATION

(71) Applicant: SS Health. Inc., Tinley Park, IL (US)

(72) Inventors: Christopher Ryan Speaker, Tinley Park, IL (US); Suzanne Alsamarrai, Tinley Park, IL (US)

(73) Assignee: SS Health, Inc., Tinley Park, IL (US)

(21) Appl. No.: 14/717,418

(22) Filed: May 20, 2015

Related U.S. Application Data
(60) Provisional application No. 62/003,324, filed on May 27, 2014.

(51) Int. Cl. A61K 33/06; C01F 7/76; A61K 9/00; A61K 45/06; A61K 9/107

(52) U.S. Cl. CPC A61K 33/06; A61K 45/06; A61K 9/107; A61K 9/0014; C01F 7/762

(57) ABSTRACT

The present invention provides a formulation for the topical application to an external portion of a human body which includes the active ingredient of potassium alum. The topical formulation is comprised of potassium alum in a predetermined weight or amount in conjunction with other ingredients to assist in the treatment

### CROSS REFERENCE

This application claims the benefit of U.S. Provisional Application No. 62/003324, filed May 27, 2014. The contents of the referenced applications are incorporated into the present application by reference.

### BACKGROUND OF THE INVENTION

The invention relates to a method of treating the medical conditions of excessive growth tissue such as granulation tissue, and “ostomies” such as gastrostomies, tracheotomies, jejunostomies, urostomies, colostomies, and ileostomies using a topical formulation comprising potassium alum, either alone or in combinations

It is well known that a patient's skin around ostomy sites can often become irritated, excoriated, and infected. The ostomy site can also develop granulation tissue which develops as a result of the ostomy closing in an attempt to heal itself. The present invention is directed at protecting the surrounding skin at ostomy sites and shrinking the unwanted granulation tissue. Current treatments for hypergranulation tissue include the use of topical steroids such as triamcinolone or application of silver nitrate. The issue with the use topical steroids, however, is that they lose their efficacy over time and often have adverse effects with long-term, or sporadic use. Likewise, there are issues with using silver nitrate over time. Silver Nitrate can be caustic, is poisonous, and can migrate onto healthy tissue. It is known that silver nitrate may “tattoo” healthy skin with every potential application.

The present invention does not suffer from such drawbacks and offers a safe and effective treatment of skin conditions associated with ostomy sites.

### SUMMARY OF INVENTION

The inventors discovered that a particular formulation for a topical treatment can effectively be used to treat “ostomies.” In the present invention, there is provided a formulation for topical application to an external portion of a human body which includes the active ingredient of potassium alum. The topical formulation is comprised of potassium alum in a predetermined weight or amount in conjunction rich other ingredients to assist in the treatment.

### DETAILED DESCRIPTION OF THE INVENTION

The present invention is directed to method of treating medical condition inherent of “ostomies” using a topical formulation(s) comprising potassium alum, either alone or in combinations.

The use of potassium alum is critical in the present invention and in treating unwanted hypergranulation tissue associated with enteral feeding tubes and ostomies. The addition of and micronization of potassium alum allows the present invention to act as an astringent. The micronized potassium alum creates a hyperosmolar state, which draws the fluid out of the granulation cells, causing them to shrink and regress

In a preferred embodiment of the present invention, the topical formulation is comprised of potassium alum in a range of 0.01%-55% by weight.

The presence of potassium alum in the present invention may be in any and all forms of potassium alum including but not limited to solubilized, micronized, dissolved, powder form, crystallized, paste, wound gel, and any other available forms of potassium alum.

The topical formulation may be further comprised of zinc oxide, menthol and lidocaine. The ingredient of zinc oxide acts as a skin barrier to protect the surrounding skin at an ostomy site, acts as an antimicrobial and also acts as an astringent. The ingredients of menthol and lidocaine act as a topical anesthetic to soothe irritated skin that is excoriated.

The formulation of the present invention may be further comprised of anti-microbial agents or ingredients such as melaleuca alterifolia, and potassium alum, for their anti-microbial properties. In one embodiment, the present invention may be comprised of silver as an antimicrobial agent as an additional agent.

In one embodiment, the topical formulation is comprised of:

Zinc Oxide from about 0% to 20%;

Menthol from about 0.1 to 4%;

Potassium Alum from about 0.1 to 55%;

Aloe Vera Gel from about 0.01-20%; and

Lidocaine HCI from about 0.01 to 7%.

The inclusion of Aloe Vera gel is for the purposes of its soothing qualities for a patient's skin. Lidocaine HCO may be included for its external analgesic qualities.

An embodiment of the present invention may further include melaleuca alterifolia, silver and potassium alum, in the amount of 0% to 2% by weight for their anti-microbial properties.

The composition may include a suitable amount of water, other solvent(s), electrolyte(s), pH modifier(s), surfactant(s), absorption enhancer(s), emulsifier(s), thickener(s), fragrant(s), preservative(s), coloring agents, or a mixture thereof.

In one embodiment, the present invention may be comprised of zinc oxide, menthol, Betaglucan, BHT, glyceryl stearate, glycrrhiza glabra (licorice) extract, lanolin, melalauca alternifolia (tea tree oil), mineral oil, PEG-100 stearate, petrolatum, polysorbate-20, potassium alum, silver, water and blue coloring.

In other embodiments of the present invention may include additional the inclusion of Collagen, Procollagen, sodium alginate, glycerin, hydroethylcellulose, Triethanolamine, benzethonium chloride, zinc acetate, polyethylene glycol, Carbomer, Citric Acid, Disodium EDTA, Glutamic Acid, Imidazolidinyl Urea, Methylparaben, Panthenol, Potassium Sorbate, PVP, Sodium Benzoate, Sodium Chloride, Sodium Metabisulfte, Water, Olive Oil, Sunflower Oil, Phenoxyethanol, Vitamin E, and Nanosilver.

The composition may include a suitable amount of water, other solvent(s), electrolyte(s), pH modifier(s), surfactant(s), absorption enhancer(s), emulsifier(s), thickener(s), fragrant(s), preservative(s), coloring agents, or a mixture thereof.

It will be appreciated by those of skill in the art that the various ingredients of the present invention may be combined all at once, or in groups, or separately.

The foregoing detailed description is given for clearness of understanding only, and no unnecessary limitations should be understood therefrom, as modifications will be obvious to those skilled in the art.

1. A topical formulation for the treatment of ostomies comprising a composition including potassium alum in a predetermined weight.

2. The topical formulation of claim 1, wherein the formulation is an emulsion.

4. The topical formulation of claim 1, wherein the formulation is a cream, lotion, or solution.

5. The topical formulation of claim 1, wherein the composition includes potassium alum from about 0.001% to about 55% by weight.

6. The topical skin composition of claim 5, wherein the composition includes anti microbial agents.

7. A method of treating skin comprising topically applying the composition of claim 1 to skin for the treatment of ostomies.
